(19) United States
(12) Patent Application Publication
(10) Pub. No.: US 2012/0136349 A1
(43) Pub. Date: May 31, 2012

(54) RENAL DENERVATION CATHETER AND METHOD USING pH ALTERATION

(76) Inventor: Roger Hastings, Maple Grove, MN (US)

(21) Appl. No.: 13/295,185

(22) Filed: Nov. 14, 2011

Related U.S. Application Data
(60) Provisional application No. 61/418,237, filed on Nov. 30, 2010.

(51) Int. Cl. A61B 18/00

(52) U.S. Cl. 606/34

(57) ABSTRACT

A catheter includes a multiplicity of leads having exposed distal elements defining an anode and a cathode positionable relative to an outer wall of a renal artery. A power supply is configured to couple to the multiplicity of leads. The power supply generates a DC current that flows between the anode and cathode to create an acidic region at the anode sufficient to cause necrosis of perivascular renal nerve tissue in the vicinity of the anode, and to create a basic region at the cathode sufficient to cause necrosis of perivascular renal nerve tissue in the vicinity of the cathode. The catheter may be configured to deliver a biocompatible electrolytic fluid to each of the cathode and anode, thereby increasing an extent of perivascular renal nerve tissue ablation in the vicinity of the cathode and anode.

### RELATED PATENT DOCUMENTS

This application claims the benefit of Provisional Patent Application Ser. No. 61/418,237 filed Nov. 30, 2010, to which priority is claimed pursuant to 35 U.S.C. §119(e) and which are hereby incorporated herein by reference.

### SUMMARY

Embodiments of the disclosure are directed to an ablation catheter having a flexible shaft with a proximal end, a distal end, a lumen arrangement extending between the proximal and distal ends, and a length sufficient to access a target vessel of the body. A multiplicity of leads extend between the proximal and distal ends of the shaft and have exposed distal elements defining an anode and a cathode positionable relative to an outer wall of the target vessel. A power supply is configured to couple to the multiplicity of leads. The power supply is configured to supply a DC current that flows between the anode and cathode to create an acidic region at the anode sufficient to cause necrosis of target tissue in the vicinity of the anode, and to create a basic region at the cathode sufficient to cause necrosis of target tissue in the vicinity of the cathode. A lumen arrangement of the catheter may be configured to deliver a biocompatible electrolytic fluid to each of the cathode and anode, thereby increasing an extent of target tissue ablation in the vicinity of the cathode and anode.

According to various embodiments, an apparatus includes a catheter having a flexible shaft with a proximal end, a distal end, a lumen arrangement extending between the proximal and distal ends, and a length sufficient to access a patient's renal artery relative to a percutaneous access location. A multiplicity of leads extend between the proximal and distal ends of the shaft and have exposed distal elements defining an anode and a cathode positionable relative to an outer wall of the renal artery. A power supply is configured to couple to the multiplicity of leads. The power supply is configured to generate a DC current that flows between the anode and cathode to create an acidic region at the anode sufficient to cause necrosis of perivascular renal nerve tissue in the vicinity of the anode, and to create a basic region at the cathode sufficient to cause necrosis of perivascular renal nerve tissue in the vicinity of the cathode.

In various embodiments, an imaging or visualization device is configured for intravascular deployment within the renal vein to facilitate positioning of the anode and cathode relative to the outer wall of the target vessel, such as a renal artery. In some embodiments, the imaging or visualization device is configured for transthoracic deployment. In other embodiments, an external imaging device, such as an ultrasound device, may be used to facilitate positioning of the anode and cathode relative to the outer wall of the target vessel, such as a renal artery.

According to various embodiments, a method involves accessing an outer wall of a target vessel, such as a patient's renal artery, and positioning a cathode and an anode relative to the target vessel. The method also involves causing a DC current to flow between the anode and cathode to create an acidic region at the anode sufficient to cause necrosis of target tissue in the vicinity of the anode, and to create a basic region at the cathode sufficient to cause necrosis of target tissue in the vicinity of the cathode. The method may further involve delivering a biocompatible electrolytic fluid to each of the anode and cathode, thereby increasing an extent of perivascular renal nerve tissue ablation proximate the anode and cathode. The method can also involve imaging or visualizing the target vessel to facilitate positioning of the anode and cathode relative to the outer wall of the target vessel.

These and other features can be understood in view of the following detailed discussion and the accompanying drawings.

### BRIEF DESCRIPTION OF THE DRAWINGS

FIG. 1 is an illustration of a right kidney and renal vasculature including a renal artery branching laterally from the abdominal aorta;

FIGS. 2A and 2B illustrate sympathetic innervation of the renal artery;

FIG. 3A illustrates various tissue layers of the wall of the renal artery;

FIGS. 3B and 3C illustrate a portion of a renal nerve;

FIG. 4 is a flow chart illustrating various processes of a renal denervation methodology in accordance with various embodiments;

FIGS. 5A and 5B illustrate an ablation catheter in accordance with various embodiments of the disclosure;

FIGS. 6A and 6B show an ablation catheter in accordance with various embodiments of the disclosure;

FIG. 7 shows an ablation system which includes an ablation catheter positioned adjacent a wall of a target renal artery and an imaging or visualization device deployed in an adjacent renal vein in accordance with various embodiments; and

FIG. 8 shows a delivery sheath dimensioned for placement within a renal vein in proximity to a target renal artery with an elongated member with a tissue piercing feature in accordance with various embodiments.

### DETAILED DESCRIPTION

Renal denervation is a procedure in which the nerves lying adjacent the renal artery are ablated to reduce sympathetic innervation of the kidney to reduce blood pressure and treat hypertension. Conventional approaches to perivascular renal nerve ablation by an radiofrequency (RF) device or cryothermal device in the renal artery have had difficulty in achieving the desired nerve ablation without damaging the artery wall.

Embodiments of the disclosure are directed to achieving necrosis of target tissue by passing DC current into the target tissue through electrodes positioned relative to the target tissue. According to various embodiments, the electrode chemistry is hydrolysis, in which hydrogen ions, H+, are generated at the anode or positive electrode, and hydroxyl ions, OH−, are generated at the cathode or negative electrode. The target tissue surrounding the anode is subject to a reduced pH, while target tissue surrounding the cathode is subject to an increased pH. Changes in pH at both the anode and cathode are sufficient to cause necrosis of the target tissue. The extent of target tissue necrosis is determined by the length of the electrodes, the current amplitude, and the duration of the treatment. Injection of a biocompatible electrolytic fluid at the anode and cathode expand the region of target tissue subjected to reduced and increased pH, respectively.

In the context of renal denervation, perivascular renal nerve tissue surrounding the anode has a reduced pH, while perivascular renal nerve tissue surrounding the cathode has an increased pH. Changes in pH at both the anode and cathode are sufficient to cause necrosis of the perivascular renal nerve tissue. Nerves adjacent the renal vein and ureters may also be ablated. An advantage of using a treatment approach according to various embodiments of the disclosure is that the ablation zone is limited to the outside of the renal artery, as there is little or no fluid exchange across the artery wall. Any ions that do cross to the interior of the renal artery will be diluted by flowing blood.

Various embodiments of the disclosure are directed to apparatuses and methods for renal denervation for treating hypertension. Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal denervation can reduce blood pressure, and may be a viable treatment option for many patients with hypertension who do not respond to conventional drugs.

The kidneys are instrumental in a number of body processes, including blood filtration, regulation of fluid balance, blood pressure control, electrolyte balance, and hormone production. One primary function of the kidneys is to remove toxins, mineral salts, and water from the blood to form urine. The kidneys receive about 20-25% of cardiac output through the renal arteries that branch left and right from the abdominal aorta, entering each kidney at the concave surface of the kidneys, the renal hilum.

Blood flows into the kidneys through the renal artery and the afferent arteriole, entering the filtration portion of the kidney, the renal corpuscle. The renal corpuscle is composed of the glomerulus, a thicket of capillaries, surrounded by a fluid-filled, cup-like sac called Bowman's capsule. Solutes in the blood are filtered through the very thin capillary walls of the glomerulus due to the pressure gradient that exists between the blood in the capillaries and the fluid in the Bowman's capsule. The pressure gradient is controlled by the contraction or dilation of the arterioles. After filtration occurs, the filtered blood moves through the efferent arteriole and the peritubular capillaries, converging in the interlobular veins, and finally exiting the kidney through the renal vein.

Particles and fluid filtered from the blood move from the Bowman's capsule through a number of tubules to a collecting duct. Urine is formed in the collecting duct and then exits through the ureter and bladder. The tubules are surrounded by the peritubular capillaries (containing the filtered blood). As the filtrate moves through the tubules and toward the collecting duct, nutrients, water, and electrolytes, such as sodium and chloride, are reabsorbed into the blood.

The kidneys are innervated by the renal plexus which emanates primarily from the aorticorenal ganglion. Renal ganglia are formed by the nerves of the renal plexus as the nerves follow along the course of the renal artery and into the kidney. The renal nerves are part of the autonomic nervous system which includes sympathetic and parasympathetic components. The sympathetic nervous system is known to be the system that provides the bodies “fight or flight” response, whereas the parasympathetic nervous system provides the “rest and digest” response. Stimulation of sympathetic nerve activity triggers the sympathetic response which causes the kidneys to increase production of hormones that increase vasoconstriction and fluid retention. This process is referred to as the renin-angiotensin-aldosterone-system (RAAS) response to increased renal sympathetic nerve activity.

In response to a reduction in blood volume, the kidneys secrete renin, which stimulates the production of angiotensin. Angiotensin causes blood vessels to constrict, resulting in increased blood pressure, and also stimulates the secretion of the hormone aldosterone from the adrenal cortex. Aldosterone causes the tubules of the kidneys to increase the reabsorption of sodium and water, which increases the volume of fluid in the body and blood pressure.

Congestive heart failure (CHF) is a condition that has been linked to kidney function. CHF occurs when the heart is unable to pump blood effectively throughout the body. When blood flow drops, renal function degrades because of insufficient perfusion of the blood within the renal corpuscles. The decreased blood flow to the kidneys triggers an increase in sympathetic nervous system activity (i.e., the RAAS becomes too active) that causes the kidneys to secrete hormones that increase fluid retention and vasorestriction. Fluid retention and vasorestriction in turn increases the peripheral resistance of the circulatory system, placing an even greater load on the heart, which diminishes blood flow further. If the deterioration in cardiac and renal functioning continues, eventually the body becomes overwhelmed, and an episode of heart failure decompensation occurs, often leading to hospitalization of the patient.

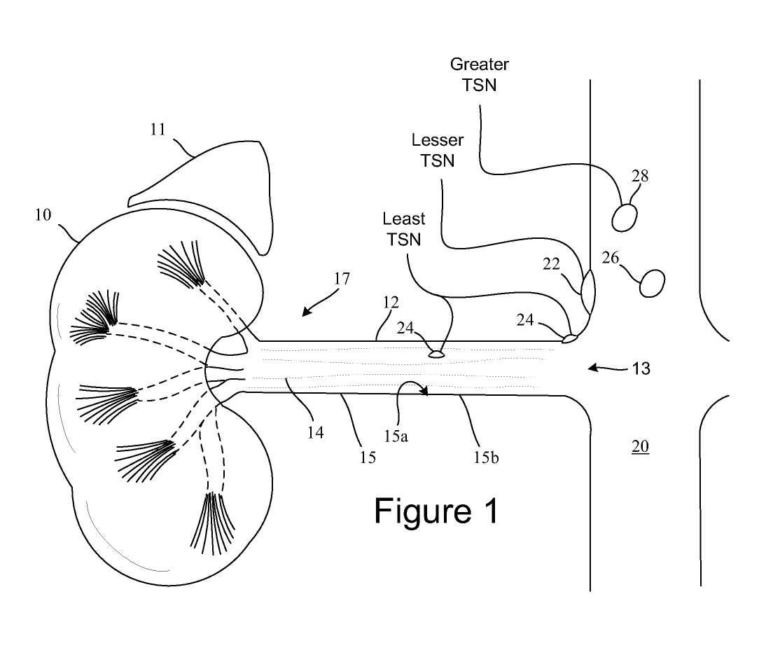

FIG. 1 is an illustration of a right kidney 10 and renal vasculature including a renal artery 12 branching laterally from the abdominal aorta 20. In FIG. 1, only the right kidney 10 is shown for purposes of simplicity of explanation, but reference will be made herein to both right and left kidneys and associated renal vasculature and nervous system structures, all of which are contemplated within the context of embodiments of the disclosure. The renal artery 12 is purposefully shown to be disproportionately larger than the right kidney 10 and abdominal aorta 20 in order to facilitate discussion of various features and embodiments of the present disclosure.

The right and left kidneys are supplied with blood from the right and left renal arteries that branch from respective right and left lateral surfaces of the abdominal aorta 20. Each of the right and left renal arteries is directed across the crus of the diaphragm, so as to form nearly a right angle with the abdominal aorta 20. The right and left renal arteries extend generally from the abdominal aorta 20 to respective renal sinuses proximate the hilum 17 of the kidneys, and branch into segmental arteries and then interlobular arteries within the kidney 10. The interlobular arteries radiate outward, penetrating the renal capsule and extending through the renal columns between the renal pyramids. Typically, the kidneys receive about 20% of total cardiac output which, for normal persons, represents about 1200 mL of blood flow through the kidneys per minute.

The primary function of the kidneys is to maintain water and electrolyte balance for the body by controlling the production and concentration of urine. In producing urine, the kidneys excrete wastes such as urea and ammonium. The kidneys also control reabsorption of glucose and amino acids, and are important in the production of hormones including vitamin D, renin and erythropoietin.

An important secondary function of the kidneys is to control metabolic homeostasis of the body. Controlling hemostatic functions include regulating electrolytes, acid-base balance, and blood pressure. For example, the kidneys are responsible for regulating blood volume and pressure by adjusting volume of water lost in the urine and releasing erythropoietin and renin, for example. The kidneys also regulate plasma ion concentrations (e.g., sodium, potassium, chloride ions, and calcium ion levels) by controlling the quantities lost in the urine and the synthesis of calcitrol. Other hemostatic functions controlled by the kidneys include stabilizing blood pH by controlling loss of hydrogen and bicarbonate ions in the urine, conserving valuable nutrients by preventing their excretion, and assisting the liver with detoxification.

Also shown in FIG. 1 is the right suprarenal gland 11, commonly referred to as the right adrenal gland. The suprarenal gland 11 is a star-shaped endocrine gland that rests on top of the kidney 10. The primary function of the suprarenal glands (left and right) is to regulate the stress response of the body through the synthesis of corticosteroids and catecholamines, including cortisol and adrenaline (epinephrine), respectively. Encompassing the kidneys 10, suprarenal glands 11, renal vessels 12, and adjacent perirenal fat is the renal fascia, e.g., Gerota's fascia, (not shown), which is a fascial pouch derived from extraperitoneal connective tissue.

The autonomic nervous system of the body controls involuntary actions of the smooth muscles in blood vessels, the digestive system, heart, and glands. The autonomic nervous system is divided into the sympathetic nervous system and the parasympathetic nervous system. In general terms, the parasympathetic nervous system prepares the body for rest by lowering heart rate, lowering blood pressure, and stimulating digestion. The sympathetic nervous system effectuates the body's fight-or-flight response by increasing heart rate, increasing blood pressure, and increasing metabolism.

In the autonomic nervous system, fibers originating from the central nervous system and extending to the various ganglia are referred to as preganglionic fibers, while those extending from the ganglia to the effector organ are referred to as postganglionic fibers. Activation of the sympathetic nervous system is effected through the release of adrenaline (epinephrine) and to a lesser extent norepinephrine from the suprarenal glands 11. This release of adrenaline is triggered by the neurotransmitter acetylcholine released from preganglionic sympathetic nerves.

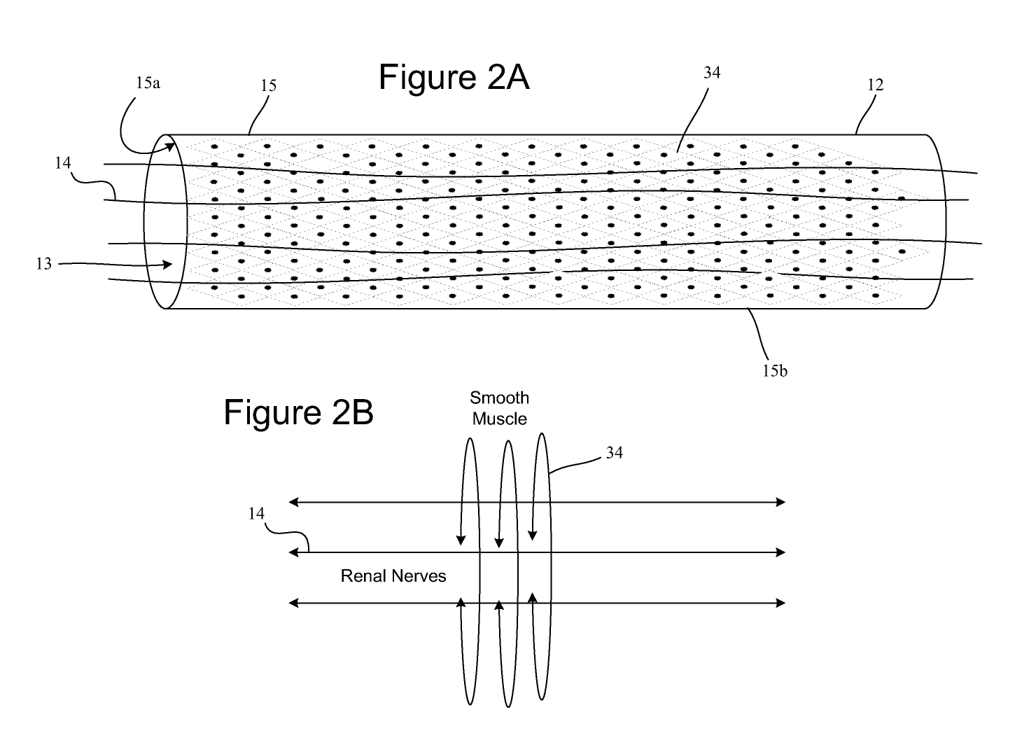

The kidneys and ureters (not shown) are innervated by the renal nerves 14. FIGS. 1 and 2A-2B illustrate sympathetic innervation of the renal vasculature, primarily innervation of the renal artery 12. The primary functions of sympathetic innervation of the renal vasculature include regulation of renal blood flow and pressure, stimulation of renin release, and direct stimulation of water and sodium ion reabsorption.

Most of the nerves innervating the renal vasculature are sympathetic postganglionic fibers arising from the superior mesenteric ganglion 26. The renal nerves 14 extend generally axially along the renal arteries 12, enter the kidneys 10 at the hilum 17, follow the branches of the renal arteries 12 within the kidney 10, and extend to individual nephrons. Other renal ganglia, such as the renal ganglia 24, superior mesenteric ganglion 26, the left and right aorticorenal ganglia 22, and celiac ganglia 28 also innervate the renal vasculature. The celiac ganglion 28 is joined by the greater thoracic splanchnic nerve (greater TSN). The aorticorenal ganglia 26 is joined by the lesser thoracic splanchnic nerve (lesser TSN) and innervates the greater part of the renal plexus.

Sympathetic signals to the kidney 10 are communicated via innervated renal vasculature that originates primarily at spinal segments T10-T12 and L1. Parasympathetic signals originate primarily at spinal segments S2-S4 and from the medulla oblongata of the lower brain. Sympathetic nerve traffic travels through the sympathetic trunk ganglia, where some may synapse, while others synapse at the aorticorenal ganglion 22 (via the lesser thoracic splanchnic nerve, i.e., lesser TSN) and the renal ganglion 24 (via the least thoracic splanchnic nerve, i.e., least TSN). The postsynaptic sympathetic signals then travel along nerves 14 of the renal artery 12 to the kidney 10. Presynaptic parasympathetic signals travel to sites near the kidney 10 before they synapse on or near the kidney 10.

With particular reference to FIG. 2A, the renal artery 12, as with most arteries and arterioles, is lined with smooth muscle 34 that controls the diameter of the renal artery lumen 13. Smooth muscle, in general, is an involuntary non-striated muscle found within the media layer of large and small arteries and veins, as well as various organs. The glomeruli of the kidneys, for example, contain a smooth muscle-like cell called the mesangial cell. Smooth muscle is fundamentally different from skeletal muscle and cardiac muscle in terms of structure, function, excitation-contraction coupling, and mechanism of contraction.

Smooth muscle cells can be stimulated to contract or relax by the autonomic nervous system, but can also react on stimuli from neighboring cells and in response to hormones and blood borne electrolytes and agents (e.g., vasodilators or vasoconstrictors). Specialized smooth muscle cells within the afferent arteriole of the juxtaglomerular apparatus of kidney 10, for example, produces renin which activates the angiotension II system.

The renal nerves 14 innervate the smooth muscle 34 of the renal artery wall 15 and extend lengthwise in a generally axial or longitudinal manner along the renal artery wall 15. The smooth muscle 34 surrounds the renal artery circumferentially, and extends lengthwise in a direction generally transverse to the longitudinal orientation of the renal nerves 14, as is depicted in FIG. 2B.

The smooth muscle 34 of the renal artery 12 is under involuntary control of the autonomic nervous system. An increase in sympathetic activity, for example, tends to contract the smooth muscle 34, which reduces the diameter of the renal artery lumen 13 and decreases blood perfusion. A decrease in sympathetic activity tends to cause the smooth muscle 34 to relax, resulting in vessel dilation and an increase in the renal artery lumen diameter and blood perfusion. Conversely, increased parasympathetic activity tends to relax the smooth muscle 34, while decreased parasympathetic activity tends to cause smooth muscle contraction.

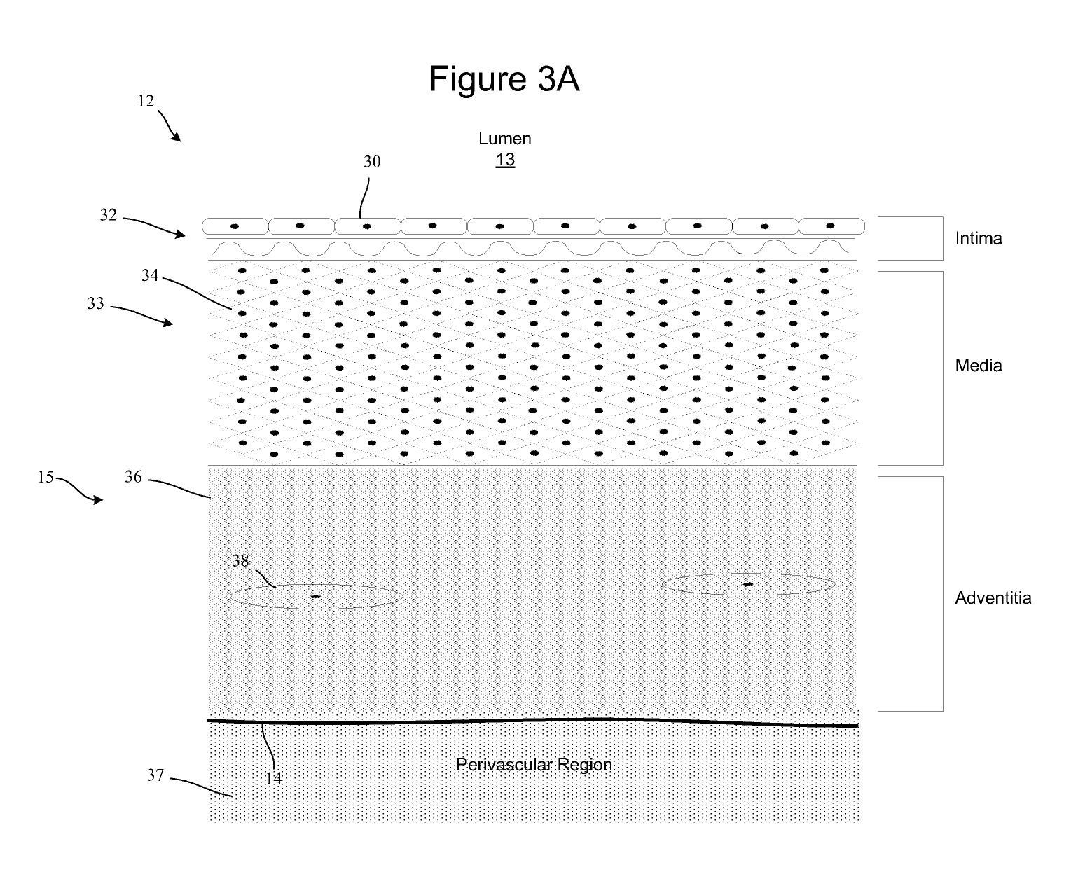

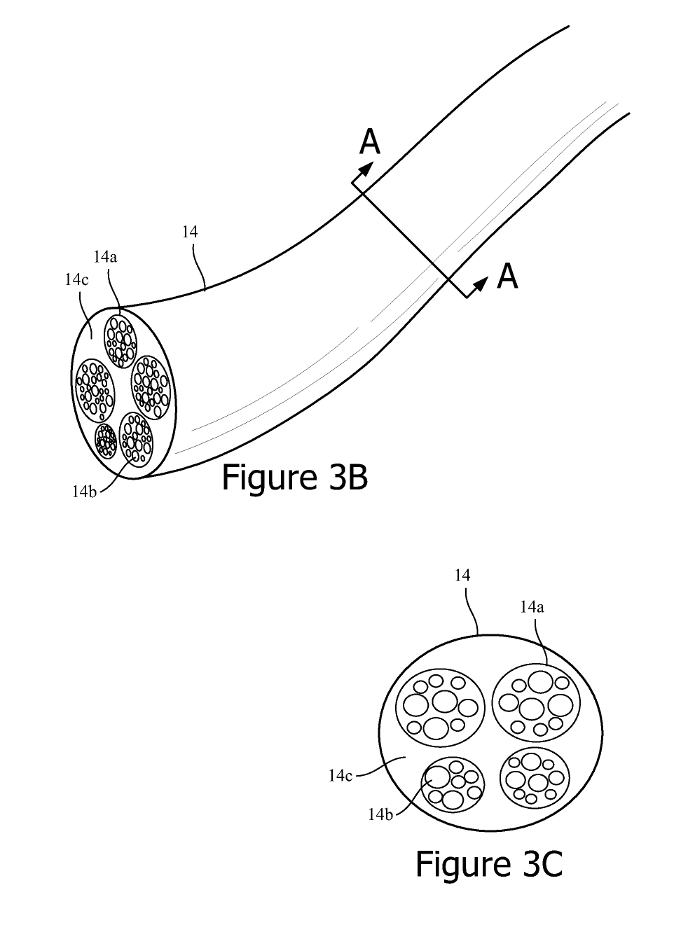

FIG. 3A shows a segment of a longitudinal cross-section through a renal artery, and illustrates various tissue layers of the wall 15 of the renal artery 12. The innermost layer of the renal artery 12 is the endothelium 30, which is the innermost layer of the intima 32 and is supported by an internal elastic membrane. The endothelium 30 is a single layer of cells that contacts the blood flowing though the vessel lumen 13. Endothelium cells are typically polygonal, oval, or fusiform, and have very distinct round or oval nuclei. Cells of the endothelium 30 are involved in several vascular functions, including control of blood pressure by way of vasoconstriction and vasodilation, blood clotting, and acting as a barrier layer between contents within the lumen 13 and surrounding tissue, such as the membrane of the intima 32 separating the intima 32 from the media 34, and the adventitia 36. The membrane or maceration of the intima 32 is a fine, transparent, colorless structure which is highly elastic, and commonly has a longitudinal corrugated pattern.

Adjacent the intima 32 is the media 33, which is the middle layer of the renal artery 12. The media is made up of smooth muscle 34 and elastic tissue. The media 33 can be readily identified by its color and by the transverse arrangement of its fibers. More particularly, the media 33 consists principally of bundles of smooth muscle fibers 34 arranged in a thin plate-like manner or lamellae and disposed circularly around the arterial wall 15. The outermost layer of the renal artery wall 15 is the adventitia 36, which is made up of connective tissue. The adventitia 36 includes fibroblast cells 38 that play an important role in wound healing.

A perivascular region 37 is shown adjacent and peripheral to the adventitia 36 of the renal artery wall 15. A renal nerve 14 is shown proximate the adventitia 36 and passing through a portion of the perivascular region 37. The renal nerve 14 is shown extending substantially longitudinally along the outer wall 15 of the renal artery 12. The main trunk of the renal nerves 14 generally lies in or on the adventitia 36 of the renal artery 12, often passing through the perivascular region 37, with certain branches coursing into the media 33 to enervate the renal artery smooth muscle 34.

Embodiments of the disclosure may be implemented to provide varying degrees of denervation therapy to innervated renal vasculature. For example, embodiments of the disclosure may provide for control of the extent and relative permanency of renal nerve impulse transmission interruption achieved by denervation therapy delivered using a treatment apparatus of the disclosure. The extent and relative permanency of renal nerve injury may be tailored to achieve a desired reduction in sympathetic nerve activity (including a partial or complete block) and to achieve a desired degree of permanency (including temporary or irreversible injury).

Returning to FIGS. 3B and 3C, the portion of the renal nerve 14 shown in FIGS. 3B and 3C includes bundles 14a of nerve fibers 14b each comprising axons or dendrites that originate or terminate on cell bodies or neurons located in ganglia or on the spinal cord, or in the brain. Supporting tissue structures 14c of the nerve 14 include the endoneurium (surrounding nerve axon fibers), perineurium (surrounds fiber groups to form a fascicle), and epineurium (binds fascicles into nerves), which serve to separate and support nerve fibers 14b and bundles 14a. In particular, the endoneurium, also referred to as the endoneurium tube or tubule, is a layer of delicate connective tissue that encloses the myelin sheath of a nerve fiber 14b within a fasciculus.

Major components of a neuron include the soma, which is the central part of the neuron that includes the nucleus, cellular extensions called dendrites, and axons, which are cable-like projections that carry nerve signals. The axon terminal contains synapses, which are specialized structures where neurotransmitter chemicals are released in order to communicate with target tissues. The axons of many neurons of the peripheral nervous system are sheathed in myelin, which is formed by a type of glial cell known as Schwann cells. The myelinating Schwann cells are wrapped around the axon, leaving the axolemma relatively uncovered at regularly spaced nodes, called nodes of Ranvier. Myelination of axons enables an especially rapid mode of electrical impulse propagation called saltation.

In some embodiments, a treatment apparatus of the disclosure may be implemented to deliver denervation therapy that causes transient and reversible injury to renal nerve fibers 14b. In other embodiments, a treatment apparatus of the disclosure may be implemented to deliver denervation therapy that causes more severe injury to renal nerve fibers 14b, which may be reversible if the therapy is terminated in a timely manner. In preferred embodiments, a treatment apparatus of the disclosure may be implemented to deliver denervation therapy that causes severe and irreversible injury to renal nerve fibers 14b, resulting in permanent cessation of renal sympathetic nerve activity. For example, a treatment apparatus may be implemented to deliver a denervation therapy that disrupts nerve fiber morphology to a degree sufficient to physically separate the endoneurium tube of the nerve fiber 14b, which can prevent regeneration and re-innervation processes.

By way of example, and in accordance with Seddon's classification as is known in the art, a treatment apparatus of the disclosure may be implemented to deliver a denervation therapy that interrupts conduction of nerve impulses along the renal nerve fibers 14b by imparting damage to the renal nerve fibers 14b consistent with neruapraxia. Neurapraxia describes nerve damage in which there is no disruption of the nerve fiber 14b or its sheath. In this case, there is an interruption in conduction of the nerve impulse down the nerve fiber, with recovery taking place within hours to months without true regeneration, as Wallerian degeneration does not occur. Wallerian degeneration refers to a process in which the part of the axon separated from the neuron's cell nucleus degenerates. This process is also known as anterograde degeneration. Neurapraxia is the mildest form of nerve injury that may be imparted to renal nerve fibers 14b by use of a treatment apparatus according to embodiments of the disclosure.

A treatment apparatus may be implemented to interrupt conduction of nerve impulses along the renal nerve fibers 14b by imparting damage to the renal nerve fibers consistent with axonotmesis. Axonotmesis involves loss of the relative continuity of the axon of a nerve fiber and its covering of myelin, but preservation of the connective tissue framework of the nerve fiber. In this case, the encapsulating support tissue 14c of the nerve fiber 14b is preserved. Because axonal continuity is lost, Wallerian degeneration occurs. Recovery from axonotmesis occurs only through regeneration of the axons, a process requiring time on the order of several weeks or months. Electrically, the nerve fiber 14b shows rapid and complete degeneration. Regeneration and re-innervation may occur as long as the endoneural tubes are intact.

A treatment apparatus may be implemented to interrupt conduction of nerve impulses along the renal nerve fibers 14b by imparting damage to the renal nerve fibers 14b consistent with neurotmesis. Neurotmesis, according to Seddon's classification, is the most serious nerve injury in the scheme. In this type of injury, both the nerve fiber 14b and the nerve sheath are disrupted. While partial recovery may occur, complete recovery is not possible. Neurotmesis involves loss of continuity of the axon and the encapsulating connective tissue 14c, resulting in a complete loss of autonomic function, in the case of renal nerve fibers 14b. If the nerve fiber 14b has been completely divided, axonal regeneration causes a neuroma to form in the proximal stump.

A more stratified classification of neurotmesis nerve damage may be found by reference to the Sunderland System as is known in the art. The Sunderland System defines five degrees of nerve damage, the first two of which correspond closely with neurapraxia and axonotmesis of Seddon's classification. The latter three Sunderland System classifications describe different levels of neurotmesis nerve damage.

The first and second degrees of nerve injury in the Sunderland system are analogous to Seddon's neurapraxia and axonotmesis, respectively. Third degree nerve injury, according to the Sunderland System, involves disruption of the endoneurium, with the epineurium and perineurium remaining intact. Recovery may range from poor to complete depending on the degree of intrafascicular fibrosis. A fourth degree nerve injury involves interruption of all neural and supporting elements, with the epineurium remaining intact. The nerve is usually enlarged. Fifth degree nerve injury involves complete transection of the nerve fiber 14b with loss of continuity.

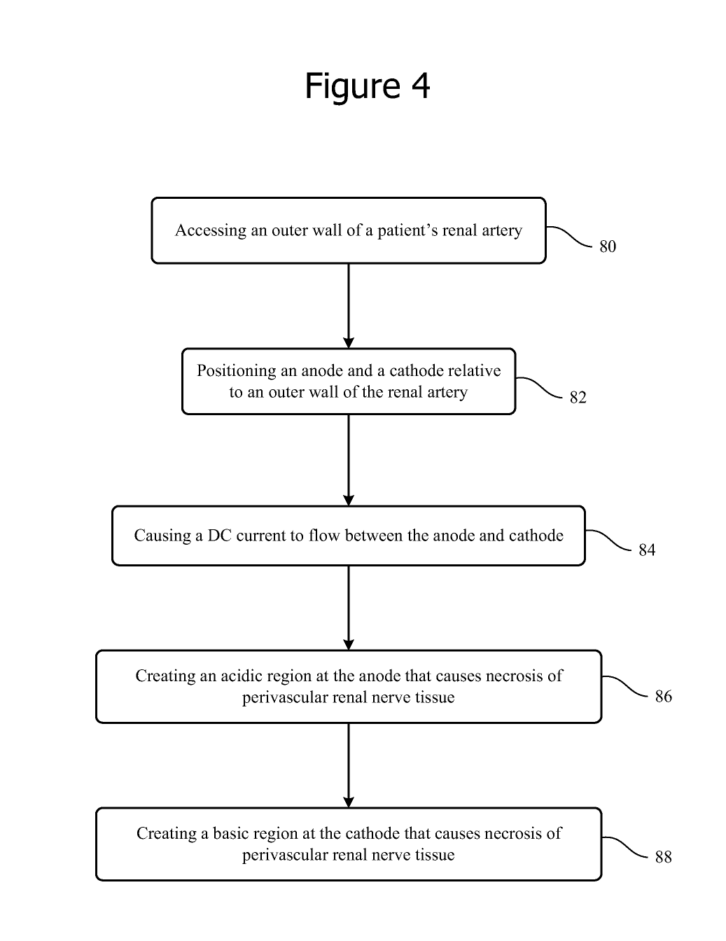

In accordance with various embodiments, and as illustrated in FIG. 4, a renal denervation method involves accessing 80 an outer wall of a patient's renal artery, and positioning 82 a cathode and an anode relative to an outer wall of the renal artery. The method also involves causing 84 a DC current to flow between the anode and cathode. The DC current flow creates 86 an acidic region at the anode sufficient to cause necrosis of perivascular renal nerve tissue in the vicinity of the anode. The DC current flow also creates 88 a basic region at the cathode sufficient to cause necrosis of perivascular renal nerve tissue in the vicinity of the cathode.

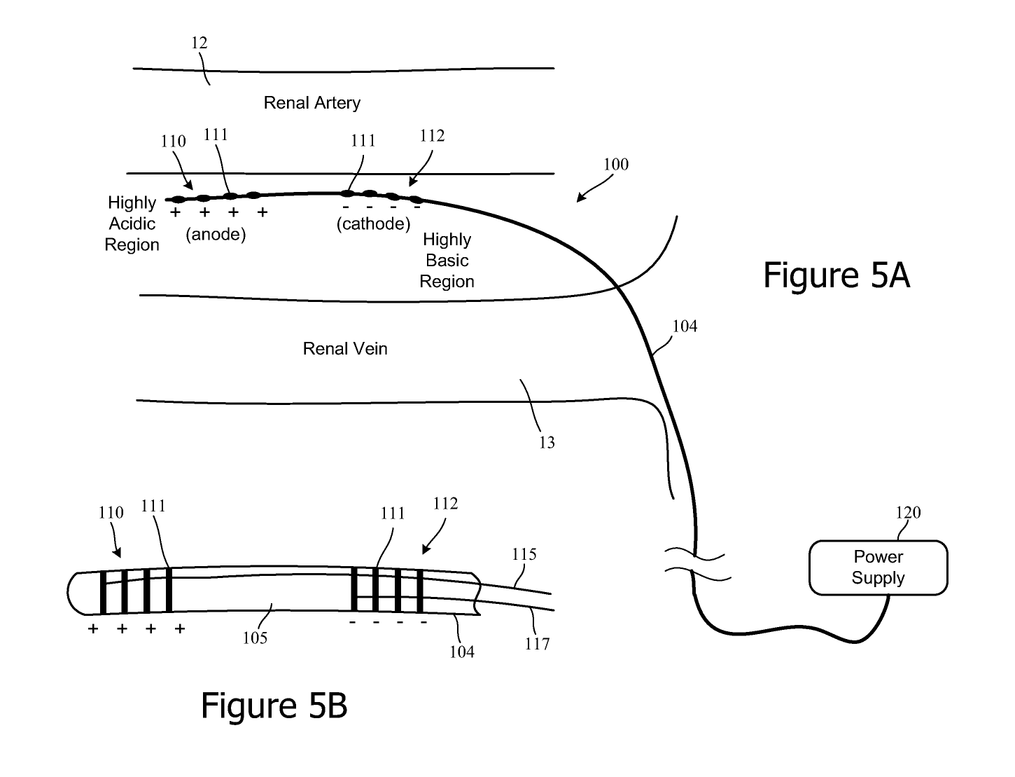

Turning now to FIGS. 5A and 5B, there is illustrated an ablation catheter 100 which includes a flexible shaft 104 having a proximal end, a distal end, a lumen arrangement 105 extending between the proximal and distal ends, and a length sufficient to access a patient's renal artery 12 relative to a percutaneous access location in accordance with various embodiments of the disclosure. The ablation catheter 100 further includes two or more leads 115 and 117 extending between the proximal and distal ends of the shaft 104 and having exposed distal elements 111 defining an anode 110 and a cathode 112 which are positionable relative to an outer wall of the renal artery 12. A power supply 120 is configured to couple to the leads 115 and 117. The power supply 120 is configured to supply a DC current that flows between the anode 110 and cathode 112 to create and acidic region at the anode 110 sufficient to cause necrosis of perivascular renal nerve tissue in the vicinity of the anode 110. The current flow also creates a basic region at the cathode 112 sufficient to cause necrosis of perivascular renal nerve tissue in the vicinity of the cathode 112. According to some embodiments, the exposed distal elements 111 define electrodes, such as ring or annular electrodes.

The DC current is passed through the tissue by ionic conduction between the anode 110 and cathode 112. In this process, tissues adjacent the anode 110 experience a pH reduction, while tissues adjacent the cathode 112 experience a pH boost. Renal nerves lying near the anode 110 or cathode 112 are ablated by the local change in pH. In some embodiments, a power supply coupled to the leads 115 and 117 can provide a constant voltage and a variable current. In other embodiments, the power supply can provide a variable voltage and a constant current.

According to some embodiments, the power supply 120 is configured to generate a constant DC current of a predetermined amplitude for a predetermined duration of time. DC current flows between the anode 110 and the cathode 112, which together define an electrochemical cell. As previously discussed, water is broken into hydrogen ions and molecular oxygen at the anode 110 or positive electrode. The H+ ions produced at the anode 110 form a highly acidic region that spreads out from the anode 110. At the cathode 112 or negative electrode, hydroxyl ions and molecular hydrogen are produced. The OH− ions produced at the cathode 112 form a highly basic region that spreads out from the cathode 112. Tissue cells are destroyed in the regions of high H+ and OH− ion concentration. The highly acidic and basic environments created at the anode and cathode cause cell necrosis of perivascular renal nerve tissue located proximate the anode and cathode, respectively.

A reduction in pH to levels between 6 and 7 at the anode and elevation in pH to levels between 8 and 9 at the cathode cause cell necrosis. For example, pH levels below 6.8 or above 8.0 are sufficient to cause cell necrosis.

According to various embodiments, the lumen arrangement 105 is configured to deliver a biocompatible electrolytic fluid to each of the anode 110 and the cathode 112, to increase the extent of perivascular renal nerve tissue ablation in the vicinity of the anode 110 and the cathode 112. For example, a biocompatible electrolytic fluid can be injected adjacent the electrodes 111 to encourage a larger zone of cell damage. Saline or other fluids may be injected adjacent the electrodes 111 to facilitate the creation of ions, and to spread the zone of ablation. The biocompatible electrolytic fluid may wick down the neural tracts to selectively impact the renal nerves.

In accordance with embodiments of the disclosure, and with continued reference to FIGS. 5A and 5B, an ablation catheter 100 containing two or more leads 115 and 117 can be delivered to a renal vein 13 located proximate a target renal artery 12. An access hole can be created in a wall of the renal vein 13 and the ablation catheter 100 can be advanced through the access hole and positioned adjacent the outside wall of the target renal artery 12. With the anode 110 and cathode 112 of the ablation catheter 100 positioned at desired locations adjacent the renal artery's outer wall, a DC current is caused to flow between the anode 110 and cathode 112 to create acidic and basic regions at the anode 110 and cathode 112 sufficient to cause necrosis of perivascular renal nerve tissue in the vicinity of the anode 110 and cathode 112.

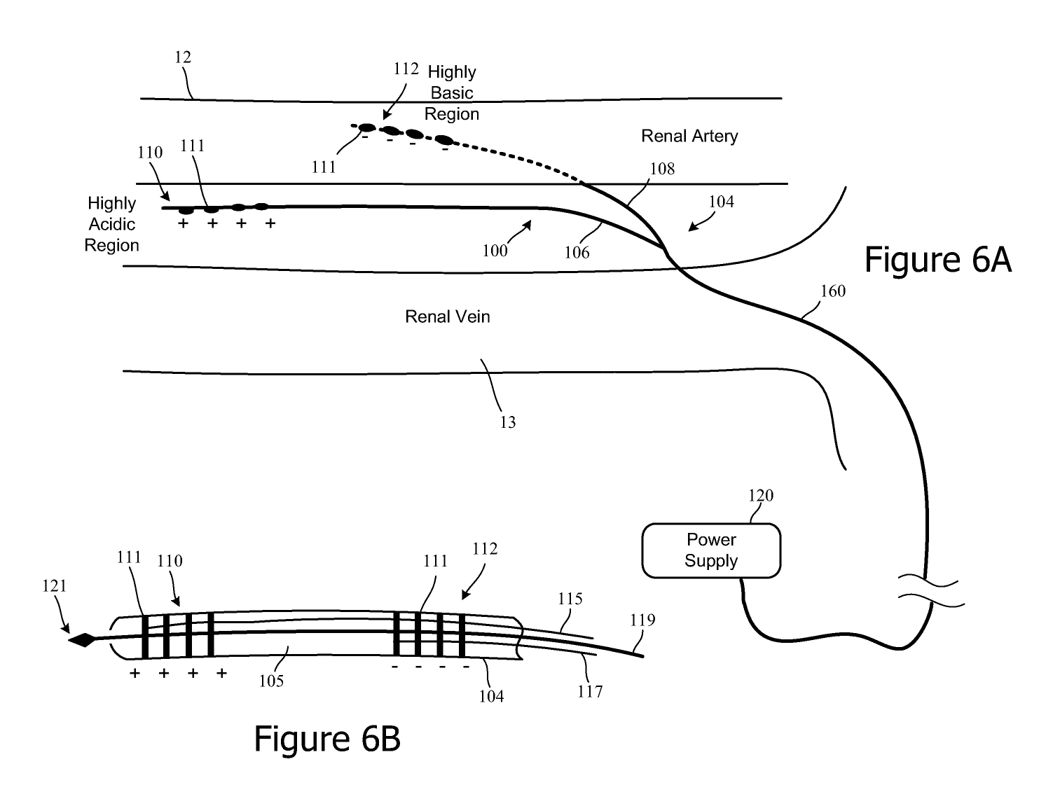

In accordance with some embodiments, and with reference to FIGS. 6A and 6B, the lumen arrangement 105 of the catheter shaft 104 may include an auxiliary lumen through which an elongated member 119 can be advanced. The distal tip of the elongated member 119 preferably includes a tissue piercing feature, such as a sharpened tip or a slightly barbed tip for creating a somewhat larger access hole. The elongated member 119 may be advanced through the auxiliary lumen and beyond a distal terminus of the shaft 104 to impinge the wall of the renal vein 13. The elongated member 119 may be further advanced to create the access hole in the wall of the renal vein 13. After creating the access hole, the elongated member 119 may be retracted from the auxiliary lumen of the catheter shaft 104. The ablation catheter 100 may then the advanced through the access hole in the renal vein 13 and to desired locations outside the wall of the renal artery 12.

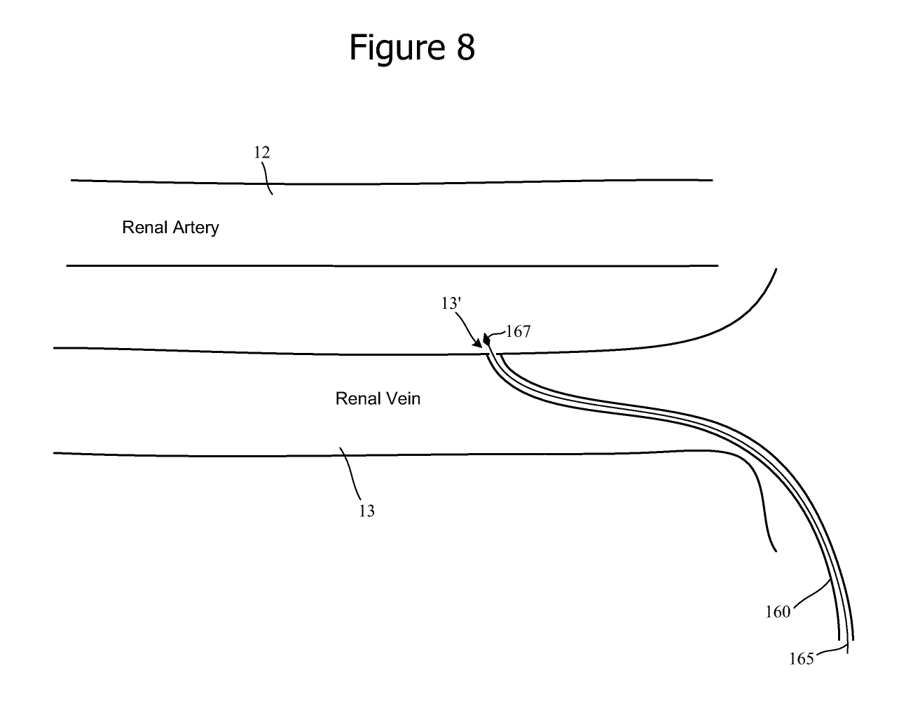

In accordance with other embodiments, and with reference to FIG. 8, a delivery sheath 160 is dimensioned for placement within a renal vein 13 in proximity to a target renal artery 12 in accordance with various embodiments. The delivery sheath 160 has a lumen dimensioned to receive an elongated member 165 having a distal tissue piercing feature 167 configured for creating an access hole in the wall of the renal vein 13. The delivery sheath lumen is also dimensioned to receive the ablation catheter 100. The elongated member 165 can be manipulated to create the access hole 13′, and then retracted from the renal vein 13 and the delivery sheath 160. The ablation catheter 100 can then be advanced through the delivery sheath 160, into the renal vein 13, through the access hole 13′, and positioned relative to the outside wall of the renal artery 12. According to some embodiments, the ablation catheter 100 can be advanced to the outer wall of the renal artery 12 via an abdominal access path, such as by use of a trocar having a lumen dimensioned to receive the ablation catheter 100.

In the embodiment illustrated in FIG. 6A, the ablation catheter 100 includes a first catheter 106 comprising at least one lead (such as lead 115 shown in FIG. 5B) and a second catheter 108 comprising at least one lead (such as lead 117 shown in FIG. 5B). The lead of the first catheter 106 includes one or more exposed elements 111 that define an anode 110. The lead of the second catheter 108 includes one or more exposed elements 111 that define a cathode 112. According to this embodiment, hydrolysis occurs in the aqueous fluid within the perivascular space between the anode 110 and cathode 112 positioned on opposing sides of the renal artery 12.

In accordance with other embodiments, the first catheter 106 shown in FIG. 6A preferably includes a multiplicity of leads having exposed elements 111 that define an anode 110 and a cathode 112. The second catheter 108 shown in FIG. 6A preferably includes a multiplicity of leads having exposed elements 111 that define an anode 110 and a cathode 112. According to this embodiment, hydrolysis occurs in the aqueous fluid within the perivascular space between anode and cathode pairs provided on each of the first and second catheters 106 and 108.

In some embodiments, the ablation catheter 100 shown in FIG. 6A may be defined by two separate catheters 106 and 108. In other embodiments, the ablation catheter 100 can include an outer sheath having a lumen dimensioned to receive the first and second catheters 106 and 108, which are displaceable within the lumen of the catheter 100. In such embodiments, the lumen of the ablation catheter 100 preferably includes a lubricious coating.

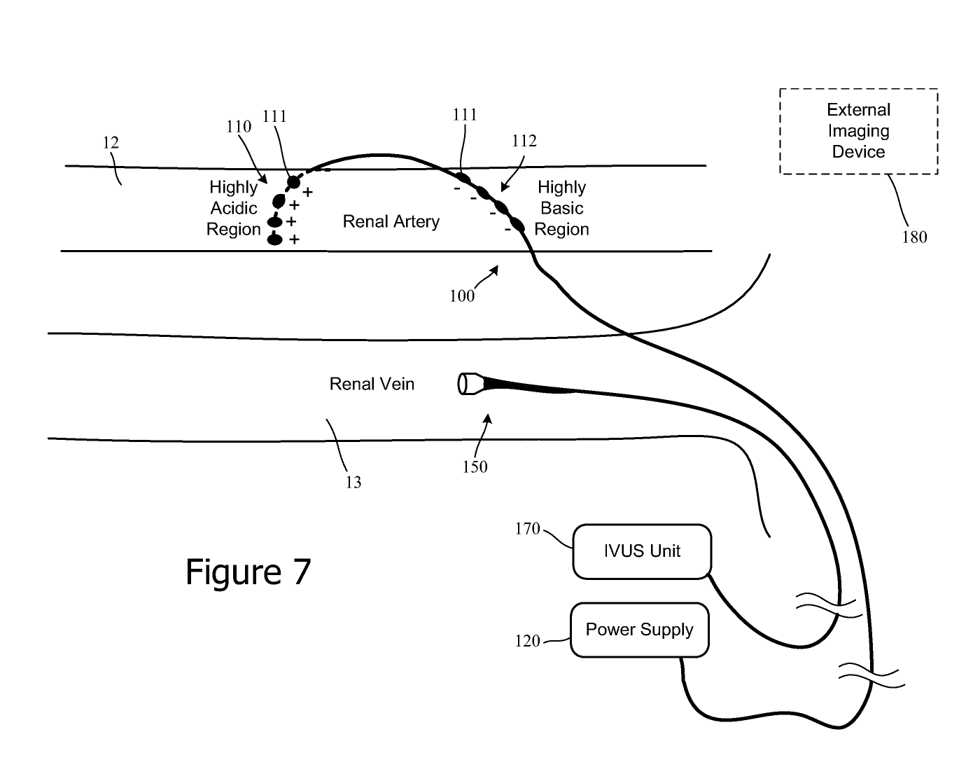

A delivery sheath 160 (shown in greater detail in FIG. 8) can be advanced into the renal vein 13 proximate the target renal artery 12 and positioned near or against the wall of the renal vein 13. An elongated member 165 having a tissue piercing tip (also shown in greater detail in FIG. 8) can be advanced through the delivery sheath 163 and access hole in the renal vein 13. After creating access hole, the elongated member 165 can be retracted from the delivery sheath 160. The ablation catheter 100 shown in FIG. 6A can then the advanced through the access hole in the renal vein 13 such that the lead of the first catheter 106 is positioned on one side of the renal artery 12 and the lead of the second catheter 108 is positioned on the other side of the renal artery 12. FIG. 7 shows an ablation catheter 100 positioned with an anode 110 and a cathode 112 positioned at desired locations adjacent a wall of a target renal artery 12 in accordance with various embodiments. According to this embodiment, an imaging or visualization device 150 (referred to herein interchangeably as “imaging device 150”) can be configured for intravascular deployment within the renal vein 13. The imaging device 150 can be an intravascular ultrasound (IVUS) device, for example. The imaging device 150 is coupled to an external system, such as an IVUS unit 170, that presents images and data to a clinician during deployment and positioning of the ablation catheter 100. The imaging device 150 and external system 170 are used to facilitate placement of the leads and exposed distal elements (e.g., electrodes 111) at desired locations adjacent the outer wall of the renal artery 12. In other embodiments, the imaging device 150 is configured for transthoracic deployment, such as through a lumen of a trocar. In further embodiments, an external imaging device 180, such as an external ultrasound device, can be used to position the leads/electrodes. In some embodiments, an endoscope can be used to facilitate placement of the leads/electrodes.

According to some embodiments, multiple catheters each having one or more electrodes can be used. In other embodiments, catheters that circle or spiral around a renal artery 12 can be used. Other embodiments involve the use of one or more catheters that exit a vessel to treat the same vessel, and one or more catheters that treat arteries, veins, or ureters.

The various embodiments disclosed herein are generally described in the context of ablation of perivascular renal nerves for control of hypertension. It is understood, however, that embodiments of the disclosure have applicability in other contexts, such as performing ablation from within other vessels of the body, including other arteries, veins, and vasculature (e.g., cardiac and urinary vasculature and vessels), and other tissues of the body, including various organs.

What is claimed is:

1. An apparatus, comprising:
a catheter comprising a flexible shaft having a proximal end, a distal end, a lumen arrangement extending between the proximal and distal ends, and a length sufficient to access a patient's renal artery relative to a percutaneous access location;
a plurality of leads extending between the proximal and distal ends of the shaft and having exposed distal elements defining an anode and a cathode positionable relative to an outer wall of the renal artery; and
a power supply configured to couple to the plurality of leads, the power supply configured to generate a DC current that flows between the anode and cathode to create an acidic region at the anode sufficient to cause necrosis of perivascular renal nerve tissue in the vicinity of the anode, and to create a basic region at the cathode sufficient to cause necrosis of perivascular renal nerve tissue in the vicinity of the cathode.

2. The apparatus according to claim 1, wherein the catheter is configured for intravascular deployment.

3. The apparatus according to claim 1, wherein the catheter is configured for transthoracic deployment.

4. The apparatus according to claim 1, wherein the catheter is dimensioned for placement within the renal vein and configured for transvascular deployment via an access hole in a wall of the renal vein.

5. The apparatus according to claim 1, wherein the catheter comprises an auxiliary lumen dimensioned to receive an elongated member comprising a tissue piercing feature at a distal tip of the elongated member.

6. The apparatus according to claim 1, comprising:
a delivery sheath dimensioned for placement within a renal vein in proximity to the patient's renal artery;
the delivery sheath configured to receive an elongated member comprising a tissue piercing feature for creating an access hole in a wall of the renal vein; and
the delivery sheath configured to receive the catheter.

7. The apparatus according to claim 1, wherein the exposed distal elements defining an anode and a cathode comprise electrodes.

8. The apparatus according to claim 1, wherein:
the catheter comprises a first catheter comprising a first plurality of the leads and a second catheter comprising a second plurality of the leads;
the first plurality of the leads extending between the proximal and distal ends of the shaft and having exposed distal elements defining an anode and a cathode positionable relative to an outer wall of the renal artery; and
the second plurality of the leads extending between the proximal and distal ends of the shaft and having exposed distal elements defining an anode and a cathode each positionable relative to an outer wall of the renal artery.

9. The apparatus according to claim 1, wherein:
the catheter comprises a first catheter comprising at least one first lead and a second catheter comprising at least one second lead;
the at least one first lead extending between the proximal and distal ends of the shaft and having one or more exposed distal elements defining one of an anode and a cathode positionable relative to an outer wall of the renal artery; and
the at least one second lead extending between the proximal and distal ends of the shaft and having one or more exposed distal elements defining the other of the anode and cathode positionable relative to an outer wall of the renal artery.

10. The apparatus according to claim 1, comprising an imaging or visualization device configured for intravascular deployment within the renal vein.

11. The apparatus according to claim 1, comprising an imaging or visualization device configured for transthoracic deployment.

12. The apparatus according to claim 1, comprising an external imaging device.

13. The apparatus according to claim 1, wherein the lumen arrangement of the catheter is configured to deliver a biocompatible electrolytic fluid to each of the cathode and anode, thereby increasing an extent of perivascular renal nerve tissue ablation in the vicinity of the cathode and anode.

14. An apparatus, comprising:
a catheter comprising a flexible shaft having a proximal end, a distal end, a lumen arrangement extending between the proximal and distal ends, and a length sufficient to access a target vessel of the body;
a plurality of leads extending between the proximal and distal ends of the shaft and having exposed distal elements defining an anode and a cathode positionable relative to an outer wall of the target vessel; and
a power supply configured to couple to the plurality of leads, the power supply configured to supply a DC current that flows between the anode and cathode to create an acidic region at the anode sufficient to cause necrosis of target tissue in the vicinity of the anode, and to create a basic region at the cathode sufficient to cause necrosis of target tissue in the vicinity of the cathode.

15. The apparatus according to claim 1, wherein the lumen arrangement of the catheter is configured to deliver a biocompatible electrolytic fluid to each of the cathode and anode, thereby increasing an extent of target tissue ablation in the vicinity of the cathode and anode.

16. A method, comprising:
accessing an outer wall of a patient's renal artery;
positioning a cathode and an anode relative to the outer wall of the renal artery; and
causing a DC current to flow between the anode and cathode to create an acidic region at the anode sufficient to cause necrosis of perivascular renal nerve tissue in the vicinity of the anode, and to create a basic region at the cathode sufficient to cause necrosis of perivascular renal nerve tissue in the vicinity of the cathode.

17. The method according to claim 16, wherein accessing the outer wall of the renal artery comprises accessing the outer wall of the renal artery via an access hole in a renal vein proximate to the renal artery.

18. The method according to claim 16, wherein accessing the outer wall of the renal artery comprises accessing the outer wall of the renal artery via a percutaneous thoracic access path.

19. The method according to claim 16, comprising delivering a biocompatible electrolytic fluid to each of the anode and cathode, thereby increasing an extent of perivascular renal nerve tissue ablation proximate the anode and cathode.

20. The method according to claim 16, comprising imaging or visualizing the renal artery to facilitate positioning of the anode and cathode relative to the outer wall of the renal artery.
